(19) United States
(12) Patent Application Publication
(10) Pub. No.: US 2013/0164800 A1
(43) Pub. Date: Jun. 27, 2013

(54) Production of Polyhydroxyalkanoates

(71) Applicants: Volodymyr Ivanov, Singapore (SG); Tet Siong Ong, Singapore (SG)

(72) Inventors: Volodymyr Ivanov, Singapore (SG); Tet Siong Ong, Singapore (SG)

(73) Assignee: G5 INTERNATIONAL HOLDINGS PTE LTD, Singapore (SG)

(21) Appl. No.: 13/634,407

(22) PCT Filed: Mar. 10, 2011
(86) PCT No.: PCT/SG2011/000094

§ 371 Date: Dec. 24, 2012

(30) Foreign Application Priority Data
Mar. 12, 2010 (GB) 1004096.2

(51) Int. Cl. C12P 7/62

(52) U.S. Cl. CPC C12P 7/625
USPC 435/135

(57) ABSTRACT

There is provided a process for producing polyhydroxyalkanoate (PHA) comprising the steps of culturing a biomass containing PHA-producing microbes in a culture media; and hydrolyzing said PHAs-producing microbes using microorganisms selected to release PHAs from the PHAs-containing microbes.

There is also provided a method of increasing the proportion of PHAs-producing microbes relative to non-PHAs producing microbes in a culture media containing both PHAs-producing microbes and non-PHAs producing microbes and a method of extracting PHAs from PHAs-containing microbes.

### TECHNICAL FIELD

The present application relates to a process for production of polyhydroxyalkanoates (PHAs).

### BACKGROUND

Polyhydroxyalkanoates (PHAs) are polyesters accumulated in biomass of many species of bacteria under growth limiting conditions. Due to their biodegradability and capability of being produced from renewable resources, PHAs can be used as alternatives to non-degradable petroleum-based plastics.

Aseptic cultivation of PHAs is employed in known processes. However, such processes require cultivation of selected or genetically recombinant strain of bacteria. Furthermore, such processes also require thermal sterilization of materials and equipment as well as specialized equipment. The cost of aseptic cultivation is several times higher than the cost of non-aseptic cultivation.

Cheap sources of carbon and energy are considered for the production of PHAs. These sources are municipal wastewater, activated sludge of municipal wastewater treatment plant, paper mill wastewater, corn-steeped liquor, molasses, activated sludge palm oil mill effluent, starch and starch-containing wastes, industrial effluents containing fatty acids. Some known processes require organic wastes to undergo acidogenesis and subsequently the organic acids produced can be polymerized by PHA-producing microbial species to form PHAs (i.e. a two-stage system).

Excess of carbon and energy source may lead to the growth of glycogen-accumulating microorganisms but this problem can be overcome by acidification of the medium with a mixture of volatile fatty acids. The fatty acids for PHAs synthesis can be produced from organic wastes. Hydrolysis and acidogenesis are usually the first steps in converting the organic wastes to fatty acids that can be further utilized by PHAs-producing bacteria. However, when different organic wastes are used for fermentation, the remaining dissolved organic substances and particles often reduces the yield of PHAs in the biomass, and also reduces the quality of PHAs produced.

In another known process, PHAs are produced through thermal gasification of organic materials with carbon monoxide and hydrogen, followed by bacterial assimilation of the gases into the cell material. However, one disadvantage of such a method is that it is usually applicable for photosynthetic bacteria only and under anaerobic conditions. Accordingly, such a method often has limited applicability with respect to the type of organic materials that can be used in the production of PHAs.

In producing PHAs, some known techniques also employ specific selection methods to select PHAs producing micro-organisms. In one known method of selecting PHAs producing micro-organisms, a feast-famine cycle is applied to a fermentation reactor, wherein the cells are first grown to a desired concentration without nutrient limitation, after which an essential nutrient is limited to allow PHA synthesis. However, such method is limited to being used in batch and semi-batch processes. Furthermore, such methods require control of the operating conditions to ensure that non-PHAs producing micro-organisms do not accumulate to undesirable levels, which may be detrimental to the growth of the resident PHAs producing micro-organisms. In such methods, during the famine phase the micro-organisms frequently also tend to alter for a long time their natural cellular behavior due to cellular starvation. This may undesirably result in the growth rate of the micro-organisms, including PHAs producing micro-organisms being adversely affected. The

PHA producing ability of the PHAs producing micro-organisms may also be negatively affected as the micro-organisms switches to a “cellular starvation mode”. In addition, if many feast-famine cycles are run, accumulation of non-producing or low-producing PHAs-producing micro-organisms may result, as mentioned above may be detrimental to the growth of the resident PHAs producing micro-organisms possibly due to high cell concentration. Furthermore, batch production requires high start-up costs due to long start-up period.

PHAs recovery also poses a technological challenge, due to the solid state of PHAs granules and cell biomass. According to the present state-of-the-art, PHA-containing biomass is processed either by extraction of a dried biomass with organic solvents of PHAs, chemically by addition of cell-destroying substances, or enzymatic. Generally, an extraction step is effected after the cells have been subjected to a treatment e.g. milling or with a reducing agent.

To avoid the use of flammable and toxic organic solvents in the chemical extraction of PHAs, other methods using safer solvents have been developed. For example, proteolytic enzymes can be used. However, not only are such processes expensive and not amenable to large scale operation, only a relatively small proportion of PHAs are obtained from the process. In other words, known techniques of PHAs extraction are relatively inefficient and do not yield desirable throughput.

In summary, current known technologies of PHAs production have several disadvantages including: (1) the need to use aseptic culture of selected or genetically modified strains that requires high expenses for sterilization of equipment and medium, as well as maintenance of aseptic conditions during biosynthesis of PHA; 2) the need to use relatively expensive nutrients such as pure mineral salts and glucose or other pure sources of carbon and energy; 3) the need to use selection techniques which are limited to batch processes and which may adversely disrupt the normal cellular behavior of PHAs producing micro-organisms and 4) the need to use expensive, flammable and toxic organic solvents or energy-consuming methods for extraction of PHAs from bacterial cells.

Furthermore, known methods of PHAs synthesis and extraction suffer from high cost or environmental pollution, and are difficult to be industrialized. Accordingly, there is a need to provide a method for the production of PHAs that overcomes, or at least ameliorates, the disadvantages mentioned above. There is also a need to provide a method for selecting PHAs producing microorganisms that overcomes or at least ameliorates, the disadvantages mentioned above. There is also a need to provide a method for extracting PHAs from PHAs producing microorganism that overcomes or at least ameliorates, the disadvantages mentioned above.

### SUMMARY

According to a first aspect, there is provided a process for producing polyhydroxyalkanoate (PHA) comprising the steps of culturing a biomass containing PHA-producing microbes in a culture media and hydrolyzing said PHAs-producing microbes using microorganisms selected to release PHAs from the PHAs-containing microbes. Advantageously, the process avoids the need to use solvents to extract the PHA, which has a number of advantages. Firstly, using microbes to extract PHA reduces the cost of production because chemical solvents do not have to be utilized and therefore purchased to obtain PHA and hence solvents are not a material cost. The disclosed process can therefore be utilized to more economically produce PHA. Secondly, the use of micro-organisms to extract PHA avoids the use of solvents and thereby avoids the production of toxic products which must be disposed of.

More advantageously, the process avoids the need to use flammable and toxic organic solvents in the extraction of PHAs. The process is capable of reducing the cost of PHA production while mitigating the negative potential environmental impact of using flammable and toxic organic solvents, thereby increasing the economic viability of PHA plastics relative to petrochemical-based plastics. In one embodiment, the culture media comprises hydrogen gas. In another embodiment, the process further comprises injecting a stream of hydrogen gas into said culture medium. The hydrogen gas is preferably substantially homogenously dispersed throughout said culture media. Injecting a stream of hydrogen gas into said culture medium increases the production yield of PHAs relative to when hydrogen gas is not injected into said culture.

In one embodiment, the culture media comprises a carbon nutrient source, such as a carbohydrate source. The presence of the carbohydrate source provides an energy source of the microorganism, allowing production of PHAs to be possible. The process may also comprise the step of maintaining the supply of hydrogen in the culture media at mass ratio in the range of from 0.01 to 0.1 of hydrogen to the mass of PHAs produced. By maintaining the specific mass ratio, it has been discovered that a relatively high level of PHAs production can be achieved. The process may also comprise the step of maintaining the supply of carbohydrates in the culture media at mass ratio in the range of from 1 to 5 of carbohydrates to the mass of PHAs produced. Also, it has been discovered that the mass ratio of carbohydrates present to the mass of PHAs produced is also crucial in maintaining a constant effective production of PHAs. The process may also comprise the step of maintaining the supply of at least one of organic acids and lipids in the culture media at mass ratio in the range of from 0.5 to 5 of organic acids to the mass of PHAs produced. Likewise, it has been discovered that the amount of organic acids or lipids is also determinant on the consistency and level of PHAs being produced.

In one embodiment, the process comprises the step of providing volatile organic compounds (VOCs) to said culture medium. In another embodiment, the process further comprises injecting a gas comprising the volatile organic compounds into said culture medium. The process may also comprise the step of maintaining the supply of gaseous volatile organic compounds in the culture media at a mass ratio in the range of from 0.5 to 5 to the quantity of PHAs produced. The mass ratio of volatile organic compounds supplied to the mass of PHAs produced is crucial in maintaining a constant effective production of PHAs. It has been surprisingly found that injecting a gas comprising volatile organic compounds into said culture medium increases the production yield of PHAs relative to when volatile organic compounds is not injected into said culture medium. The addition of volatile organic compounds into the culture medium enhances the synthesis of PHAs by introducing substituents in the side chains. Advantageously, the process employing the use of volatile organic compounds may be used for a wide range of microorganisms and under aerobic conditions, which are more effective for bacterial growth and production of PHAs than anaerobic conditions. The volatile organic compounds may provide a source of carbon and energy for the PHAs production. Advantageously, the supply of VOCs in the gaseous phase to the culture media provides a higher purity of the VOCs since VOCs in the liquid phase may comprise of impurities which are absent in the gaseous phase of the VOCs.

In one embodiment, the process comprises the step of maintaining the pH of the culture media in the range of 6 to 8. Advantageously, this maintenance of the pH in the specified range contributes to the overall increase in production of PHAs. The step of maintaining the pH of the culture media may comprise providing an organic acid to said culture media.

In one embodiment, the process comprises the step of maintaining generally aerobic conditions in said culture media during said culturing step. The culture media may comprise 0.1 mgL⁻¹ to 1 mgL⁻¹ of dissolved oxygen during said culturing step.

In another embodiment, the process comprises the step of maintaining the culture media at 100 mgL⁻¹ to 1000 mgL⁻¹ of organic carbon during said culturing step.

The process may also include before said culturing step, the step of fermenting a population of PHA-producing microbes in a microbial fermentation zone. The microbial fermentation zone may be charged with a natural source of microbes that contain PHA-producing microbes. The microbes or microorganisms that may be used in said fermentation zone for the production of volatile organic compounds and hydrogen include but are not limited to the species of the genera Acetobacter, Bacteroides, Clostridium, Citrobacter, Enterobacter, Moorella, Propionibacterium, Ruminococcus, Thermoanaerobium.

In one embodiment, the microbial fermentation zone is maintained at substantially anaerobic conditions. In one embodiment, the process comprises the step of producing hydrogen gas in said microbial fermentation zone. Advantageously, the hydrogen produced can used for injection into the culture medium as described above.

In one embodiment, said fermentation zone comprises of a liquid phase and a vapor phase. In another embodiment, the process comprises the step of obtaining the volatile organic compounds from said microbial fermentation zone. In one embodiment, the obtaining step comprises the step of removing a vapor phase adjacent to or above said fermentation zone. Advantageously, the volatile organic compounds produced can be used for injection into the culture medium as described above. The microbial fermentation zone may be maintained at a pH in the range of 5 to 8. The microbial fermentation zone may also be maintained at a reduction potential of from −50 mV to −400 mV.

According to a second aspect, there is provided a method of increasing the proportion of PHAs-producing microbes relative to non-PHAs producing microbes in a culture media containing both PHAs-producing microbes and non-PHAs producing microbes, the method comprising the step of (a2) incubating said culture media in a selection zone under conditions to enable faster propagation of the PHAs-producing microbes relative to the non-PHAs producing microbes for a period of time to produce a culture media having more PHAs microbes relative to non-PHAs producing microbes. Advantageously, the method provides for positive selection, wherein PHAs-producing microbes are “selected” by virtue of their prolific growth rate. Even more advantageously, the method does not lead to adverse changes in cellular behavior of the microbes caused by cellular starvation. This allows PHAs-producing microbes to continue growing, multiplying and producing PHAs normally without any appreciable decline in overall growth rate or PHAs producing rate. The PHAs-producing microorganisms include but are not limited to the species of the genera Acinetobacter, Alcaligenes, Alcanivorax, Azotobacter, Bacillus, Burkholderia, Delftia, Klebsiella, Marinobacter, Pseudomonas, Ralstonia, Rhisobium.

In one embodiment, prior to step (a2), the method further comprises the step of (a1) providing a carbon source in the culture medium to increase the store of PHAs present in the PHAs producing microbes. Advantageously, this step enables rapid propagation of PHAs producing microbes as well as restores and increases the PHAs stores within these micro-organisms.

In another embodiment, the providing step (a1) and incubating step (a2) are carried out in separate chambers. This beneficially enables the providing step (a1) and incubating step (a2) to be carried out simultaneously in two different sets of operating conditions. Advantageously, this allows the method to be adopted in a continuous process, saving large amount of operation time and increasing the overall efficiency of the process.

In one embodiment, the method further comprises the step of (a3) passing the culture media from the chamber where the incubating step (a2) takes place, back to the other chamber where the providing step (a1) takes place. This step allows the “positively selected” PHAs-producing microbes to assimilate carbon source rapidly in the presence of a carbon source, thereby overall producing an increased amount of PHAs. Preferably, the steps (a1), (a2) and (a3) take place continuously. Advantageously, this enables the cycling of the culture media between the providing step and incubating step continuously allowing rapid “positive selection” as well as the production of PHAs to take place rapidly. More advantageously, this method can be incorporated into a continuous process and is not limited for use in a batch process, thereby increasing the overall throughput.

In one embodiment, the residence time of the culture media in the chamber where the providing step (a1) takes place is from 6 to 24 hours. The residence time of the culture media in the chamber where the incubating step (a2) takes place may be from 0.5 to 2 hours. In one embodiment, the ratio of the residence time of the culture media in the chamber where the providing step (a1) takes place to the residence time of the culture media in the chamber where the incubating step (a2) takes place is from 5 to 15. The dissolved oxygen in the incubating step (a2) may also be maintained at 1 mgL⁻¹ to 10 mgL⁻¹. Advantageously, the above conditions contribute to the overall success and effectively of the method.

According to a third aspect, there is provided a method of extracting PHAs from PHAs-containing microbes, the method comprising the steps of hydrolyzing the cell walls of the PHAs-containing microbes to release PHAs from the PHAs-containing microbes; and separating said released PHAs from said microbes. Advantageously, the method is a cost effective and efficient method of extracting the PHAs from PHAs-containing microbes.

In one embodiment, the hydrolyzing step comprises the step of using microorganisms that release enzymes that hydrolyzes bacterial cell walls. The microorganisms that release enzymes that hydrolyze bacterial cell walls may include fungi selected from the group consisting of fungi from the genera Absidia, Agaricus, Aspergillus, Chaetomium, Fusarium, Neurospora, Penicillium, Phanerophaete, Phialophora, Pleurotus, Rhizoctonia and Trichoderma.

In one embodiment, the hydrolysing step is performed simultaneously with the separating step to thereby reduce the time of contact between the hydrolytic enzymes and said released PHAs. Advantageously, this reduces the possibility of PHAs being undesirably hydrolysed by the hydrolytic enzymes. The separating step may comprise using at least one of flotative separation, centrifugation or membrane separation.

According to a fourth aspect, there is provided a process for producing polyhydroxyalkanoates (PHAs) comprising the steps of (a) incubating a culture media containing PHAs-producing microbes and non-PHAs producing microbes in a selection zone under conditions to enable faster propagation of the PHAs-producing microbes relative to the non-PHAs producing microbes, wherein said incubating is undertaken for a period of time to produce culture media having more PHA microbes relative to the non-PHA producing microbes; (b) providing said culture media produced in said incubating step (a) to a culturing zone to culture the PHA-producing microbes in a culture media; (c) providing said PHA produced in said providing step (a) to an extraction zone in which said PHA producing microbes are hydrolyzed by microorganisms to release PHAs from the PHAs-containing microbes; and (d) isolating said PHAs from said culture media.

In one embodiment, at least one of steps (a) to (d) in the process is carried out under non-aseptic conditions. In another embodiment, the process further comprises the step of returning a portion of the PHA-producing microbes produced in step (b) to the selection zone.

### DEFINITIONS

The following words and terms used herein shall have the meaning indicated:

The term “polyhydroxyalkanoate” (PHA), as used in the context of the present specification, refers broadly to renewable, thermoplastic, aliphatic polyesters and/or co-polyesters, which may be produced by polymerization of the respective monomer hydroxy aliphatic acids (including dimers of the hydroxy aliphatic acids), by bacterial fermentation of starch, sugars, lipids, etc. PHA polymers may include poly-beta-hydroxybutyrate (PHB) (also known as poly-3-hydroxybutyrate), poly-alpha-hydroxybutyrate (also known as poly-2-hydroxybutyrate), poly-3-hydroxypropionate, poly-3-hydroxyvalerate, poly-4-hydroxybutyrate, poly-4-hydroxyvalerate, poly-5-hydroxyvalerate, poly-3-hydroxyhexanoate, poly-4-hydroxyhexanoate, poly-6-hydroxyhexanoate, polyhydroxybutyrate-valerate (PHBV), polyglycolic acid, polylactic acid (PLA), etc., as well as PHA copolymers, blends, mixtures, combinations, thereof.

The term “biomass”, as used in the context of the present specification, may include natural PHA-producing bacteria, transgenic PHA-producing bacteria or mixtures thereof. In addition, said biomass may include mixtures of different varieties of PHA-producing bacteria, for example, mixed cultures of Escherichia coli and Aeromonas hydrophilia. Biomass may also include mixtures of plant biomass, bacterial biomass, and/or any other type of PHA-containing biomass.

The term “non-aseptic”, as used in the context of the present specification, refers to substantially no sterilization and disinfection of the medium and equipment, so accepting presence of microorganisms other that those of interest. This may also encompass non-pure cultures of microorganisms of interest. Likewise, the term “aseptic” should be construed accordingly.

The term “anaerobic”, as used in the context of the present specification, refers to conditions whereby an electron acceptor such as oxygen, nitrates and/or sulfates are completely absent, or substantially absent.

The term “aerobic”, as used in the context of the present specification, refers to conditions whereby there is present at least one terminal electron acceptor such as oxygen, nitrates and/or sulfates.

The term “batch process”, as used in the context of the present specification, refers to a process wherein all or at least a portion of the reactants are added to a reactor and then proceeds according to a predetermined course of reaction, during which no product is removed from the reactor.

The term “continuous process”, as used in the context of the present specification, refers to a process wherein reactants are continually introduced and products withdrawn simultaneously in an uninterrupted manner when in use or in operation.

The term “nutrients” in the context of this specification is understood to comprise any substance that allows or contributes the growth of the micro-organism.

The term “microbes” generally refers to, for example, microorganisms such as bacteria, fungi, viruses, like biological entities and combinations thereof. The terms microbes and microorganisms will be used interchangeably herein.

The term “natural source” refers to a material that occurs in the natural environment, and may comprise one or more biological entities. For example, a natural source of micorganisms can be obtained from soil, waste water and food waste.

The term “volatile organic compound” (“VOC”) as used herein shall be given their ordinary meaning and shall include, but not be limited to, highly evaporative, carbon-based chemical substances; chemical compounds that evaporate readily at room temperature and contain carbon; and/or compounds comprising carbon which participate in atmospheric photochemical reactions. The VOCs may be found from the vapor phase of an enclosed fermentation chamber that contains a population of microbes that produces PHAs. Typical volatile organic compounds include alcohols and fatty acids, particularly low carbon number alcohols and fatty acids.

Unless specified otherwise, the terms “comprising” and “comprise”, and grammatical variants thereof, are intended to represent “open” or “inclusive” language such that they include recited elements but also permit inclusion of additional, unrecited elements.

As used herein, the term “about”, in the context of concentrations of components of the formulations, typically means +/−5% of the stated value, more typically +/−4% of the stated value, more typically +/−3% of the stated value, more typically, +/−2% of the stated value, even more typically +/−1% of the stated value, and even more typically +/−0.5% of the stated value.

Throughout this disclosure, certain embodiments may be disclosed in a range format. It should be understood that the description in range format is merely for convenience and brevity and should not be construed as an inflexible limitation on the scope of the disclosed ranges. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

### Disclosure of Optional Embodiments

Exemplary, non-limiting embodiments of process for producing polyhydroxyalkanotae (PHAs), a method of increasing the proportion of PHAs-producing microbes relative to non-PHAs producing microbes in a culture media containing both PHAs-producing microbes and non-PHAs producing microbes, and a method of extracting PHAs from PHAs-containing microbes will now be disclosed.

The process for producing polyhydroxyalkanotae (PHAs) comprises the steps of culturing a biomass containing PHA-producing microbes in a culture media and providing said biomass to an extraction zone in which said PHA-producing microbes are hydrolyzed by microorganisms to release PHAs from the PHAs-containing microbes. The hydrolyzing step comprises using microorganisms that release enzymes that hydrolyzes bacterial cell walls. The microorganisms that release enzymes that hydrolyzes bacterial cell walls may be fungi selected from the group consisting of fungi from the genera Absidia, Agaricus, Aspergillus, Chaetomium, Fusarium, Neurospora, Penicillium, Phanerophaete, Phialophora, Pleurotus, Rhizoctonia and Trichoderma.

In one embodiment, the process for producing PHAs comprises the step of culturing a biomass containing PHA-producing microbes in a culture media containing hydrogen. In one embodiment, the process further comprises the step of injecting a stream of hydrogen gas into said culture medium. In one embodiment, the biomass contains PHA-producing bacteria selected from the genera consisting of Acinetobacter, Alcaligenes, Alcanivorax, Azotobacter, Bacillus, Burkholderia, Delftia, Klebsiella, Marinobacter, Pseudomonas, Ralstonia, Rhisobium.

In one embodiment, the culture media comprises volatile organic compounds (VOCs). The VOCs may comprise one or more of the following: volatile fatty acids and alcohols.

In one embodiment, the culture media comprises a carbon nutrient source, such as carbohydrates. The method may also comprise the step of maintaining the supply of hydrogen in the culture media at mass ratio in the range of from about 0.01 to about 0.1, from about 0.02 to about 0.09, from about 0.03 to about 0.08, from about 0.04 to about 0.07 or from about 0.05 to about 0.06 of hydrogen to the quantity of PHAs produced. Preferably, the mass ratio of hydrogen to the quantity of PHAs produced is about 0.02. In another embodiment, the process comprises the step of maintaining the supply of carbohydrates in the culture media at mass ratio in the range of from about 1 to about 5, from about 2 to about 4 or from about 2 to about 3 of carbohydrates to the quantity of PHAs produced. Preferably, the mass ratio of carbohydrates to the quantity of PHAs produced is about 2. The process may also comprise the step of maintaining the supply of organic acids or lipids in the culture media at mass ratio in the range of from about 0.5 to about 5, from about 1 to about 4.5, from about 1.5 to about 4, from about 2 to about 3.5 or from about 2.5 to about 3 of organic acids or lipids to the quantity of PHAs produced. Preferably, the mass ratio of organic acids or lipids to the quantity of PHAs produced is about 1.

In one embodiment, the process comprises the step of maintaining the supply of volatile organic acids in the culture media at mass ratio in the range of from about 1 to about 5, from about 2 to about 4 or from about 2 to about 3 of volatile organic acids to the quantity of PHAs produced. Preferably, the mass ratio of volatile organic acids to the quantity of PHAs produced is about 2.

The process may also comprise the step of maintaining the pH of the culture media in the range of from about 6 to about 8, from about 6.5 to about 7.5 or from about 6.5 to about 7. In one embodiment, the step of maintaining the pH of the culture media comprises providing an organic acid to said culture media. The process may also include the step of maintaining generally aerobic conditions in said culture media during said culturing step.

In one embodiment, the process the culture media comprises from about 0.1 mgL⁻¹ to about 1 mgL⁻¹, from about 0.2 mgL⁻¹ to about 0.9 mgL⁻¹, from about 0.3 mgL⁻¹ to about 0.8 mgL⁻¹, from about 0.4 mgL⁻¹ to about 0.7 mgL⁻¹, or from about 0.5 mgL⁻¹ to about 0.6 mgL⁻¹ of dissolved oxygen during said culturing step. Preferably, the culture media comprises about 0.5 mgL⁻¹ of dissolved oxygen during said culturing step

In another embodiment, the process comprises the step of maintaining the culture media at from about 100 mgL⁻¹ to about 1000 mgL⁻¹, from about 200 mgL⁻¹ to about 900 mgL⁻¹, from about 300 mgL⁻¹ to about 800 mgL⁻¹, from about 400 mgL⁻¹ to about 700 mgL⁻¹, or from about 500 mgL⁻¹ to about 600 mgL⁻¹ of organic carbon during said culturing step. Preferably, about 500 mgL⁻¹ of organic carbon is maintained in the culture media.

In one embodiment, hydrogen gas in the culturing zone is substantially homogenously dispersed throughout said culture media. The hydrogen gas may be substantially homogenously vertically and horizontally dispersed throughout the culture media.

In one embodiment, gaseous volatile organic compounds in the culturing zone are substantially homogenously dispersed throughout said culture media. The gaseous volatile organic compounds may be substantially homogenously vertically and horizontally dispersed throughout the culture media.

In one embodiment, wherein before said culturing step, the process comprises the step of fermenting a population of PHA-producing microbes in a microbial fermentation zone. The microbial fermentation zone may be charged with a natural source of microbes that contain PHA-producing microbes. In one embodiment, fermentation of organic compounds is carried out in microbial fermentation zone. The fermented organic compounds may include at least one of carbohydrates, liquid and solid lipids, microbial biomass and waste organic compounds fermented by bacteria selected from the genera consisting but are not limited toof Acetobacter, Bacteroides, Clostridium, Citrobacter, Enterobacter, Moorella, Propionibacterium, Ruminococcus, Thermoanaerobium. The mass ratio of supplied fermented carbohydrates and fermented microbial biomass in microbial fermentation zone may be maintained at from about 1 to about 10, from about 2 to about 9, from about 3 to about 8, from about 4 to about 7, from about 5 to about 6. Preferably the mass ratio of supplied fermented carbohydrates and fermented microbial biomass in microbial fermentation zone is maintained at 3.

In one embodiment, the microbial fermentation zone is maintained at substantially anaerobic conditions. The microbial fermentation zone may be inoculated with a natural source of anaerobic microorganisms. In one embodiment, the natural source of anaerobic microorganisms is soil, bottom sediments of aquatic systems, or anaerobic sludge.

The process may include the step of producing hydrogen gas in said microbial fermentation zone. The process may include the step of producing gaseous volatile organic compounds in said microbial fermentation zone. The volatile organic compounds may be extracted from the microbial fermentation zone and transferred to said culturing zone. Typically, the fermentation zone is contained within an enclosed vessel with a liquid fermentation phase and a volatile phase above it that is contained within a chamber. Accordingly, the volatile organic compounds may be extracted from the chamber by applying a vacuum from which they are then passed to the a culture media for culturing PHA-producing microbes. In one embodiment, the process comprises the step of maintaining the microbial fermentation zone at a pH in the range of 5 to 8. The process may also comprise the step of maintaining the microbial fermentation zone at a reduction potential of from about −50 mV to about −400 mV, from about −100 mV to about −350 mV, from about −150 mV to about −300 mV or from about −200 mV to about −250 mV.

In another embodiment, wherein after said culturing step, the process comprises the step of extracting PHA from said cultured PHA-producing microbes. In one embodiment, the extraction step comprises the step of hydrolyzing the PHA-producing microbes to release said PHAs. The step of hydrolyzing may comprise the use of using microorganisms that release enzymes that hydrolyzes bacterial cell walls. In one embodiment, the microorganisms that release enzymes that hydrolyzes bacterial cell walls are fungi selected from the group consisting of fungi from the genera Absidia, Agaricus, Aspergillus, Chaetomium, Fusarium, Neurospora, Penicillium, Phanerophaete, Phialophora, Pleurotus, Rhizoctonia and Trichoderma.

In one embodiment, wherein before said hydrolyzing step, said PHA-producing microbes are substantially separated from the biomass. The extraction step may be undertaken in an extraction zone at generally acidic conditions. The acidic conditions of said extraction zone can be at a pH within the range of from about 2 to about 5 or from about 3 to about 4.

In one embodiment, the process further comprises the step of separating said released PHAs from said microbes. The extracting step may also be performed simultaneously with the separating step to thereby reduce the time of contact between the hydrolytic enzymes and said released PHAs. In one embodiment, the process comprises the step of introducing an oxidant, such as hydrogen peroxide or sodium hypochlorate to said extraction zone. The treatment of bacterial biomass to dissolve bacterial cell wall and non-PHA polymers may be with a solution of sodium hypochlorate from about 0.5 to about 2% (w/v) or from about 1 to about 1.5% % (w/v). The treatment of bacterial biomass to dissolve bacterial cell wall and non-PHA polymers with sodium hypochlorate may be carried out from about 1 to about 8 hours, from about 2 to about 7 hours, from about 3 to about 6 hours or from about 4 to about 5 hours. The treatment of bacterial biomass to dissolve bacterial cell wall and non-PHA polymers with a solution of sodium hypochlorate may also be carried out at pH of from about 11 to about 12 or from about 10.5 to about 11.5.

In one embodiment, the supply of carbohydrates, organic acids, lipids, nutrients disclosed herein may be from at least one of the following sources: reject water of municipal wastewater treatment plants, glucose or glucose-containing wastes, sugar or sugar-containing wastes, lactose or lactose-containing waste such as cheese whey, acetate, vinegar or acetate-containing waste, valeric acid or valerate-containing wastes, stevia extracts, rebaudioside A (RA) stevia extract, cassava starch, corn starch, potato starch and starch-containing wastes, palm oil, vegetable oil, other wastewater from processing plant of tomatoes, potatoes, cheese, soya bean, vegeatable oil, sugar cane (molasses) and food waste, solid organic waste, waste biomass from municipal or industrial wastewater treatment plant.

The method of increasing the proportion of PHAs-producing microbes relative to non-PHAs producing microbes in a culture media containing both PHAs-producing microbes and non-PHAs producing microbes comprises the step of (a2) incubating said culture media in a selection zone under conditions to enable faster propagation of the PHAs-producing microbes relative to non-PHAs producing microbes for a period of time to produce a culture media having more PHA microbes relative to non-PHA producing microbes. In one embodiment, the conditions in the selection zone apart from enabling faster propagation of the PHAs-producing microbes relative to non-PHAs producing microbes, does not substantially lead to adverse physiological changes in cellular behavior of the microbes caused by cellular starvation.

In one embodiment, wherein prior to step (a2), the method further comprises the step of (a1) providing said culture media with a carbon source to increase the store of PHAs present in the PHAs producing microbes. The feeding step (a1) and incubating step (a2) may be carried out in separate chambers.

The method may further comprise the step of (a3) passing the culture media from the chamber where the incubating step (a2) takes place, back to the other chamber where the feeding step (a1) takes place. In one embodiment, the steps (a1), (a2) and (a3) take place continuously.

The residence time of the culture media in the chamber where the feeding step (a1) takes place may be from about 6 to about 24 hours, from about 8 to about 22 hours, from about 10 to about 20 hours, from about 12 to about 18 hours or from about 14 to about 16 hours. The residence time of the culture media in the chamber where the where the incubating step (a2) takes place may be about from about 0.5 to about 2 hours or from 1 to about 1.5 hours. The ratio of the residence time of the culture media in the chamber where the feeding step (a1) takes place to the residence time of the mixture in the chamber where the incubating step (a2) takes place may be from about 5 to about 10, from about 6 to about 10, from about 7 to about 10, from about 8 to about 10, or from about 9 to about 10.

In one embodiment, wherein the dissolved oxygen in the incubating step (a2) is maintained at from about 1 mgL⁻¹ to about 10 mgL⁻¹, from about 2 mgL⁻¹ to about 9 mgL⁻¹, from about 3 mgL⁻¹ _{to about} 8 mgL⁻¹, from about 4 mgL⁻¹ to about 7 mgL⁻¹, from about 5 mgL⁻¹ to about 6 mgL^{-1.} Preferably the dissolved oxygen in the incubating step (a2) is maintained at 3 mgL⁻¹.

The method of extracting PHAs from PHAs-containing microbes, the method comprising the steps of hydrolyzing the cell walls of the PHAs-containing microbes to release PHAs from the PHAs-containing microbes; and separating said released PHAs from said microbes.

In one embodiment, the hydrolyzing step comprises using microorganisms that release enzymes that hydrolyzes bacterial cell walls. The microorganisms that release enzymes that hydrolyzes bacterial cell walls may be fungi selected from the group consisting of fungi from the genera Absidia, Agaricus, Aspergillus, Chaetomium, Fusarium, Neurospora, Penicillium, Phanerophaete, Phialophora, Pleurotus, Rhizoctonia and Trichoderma. The hydrolysing step may be performed simultaneously with the separating step to thereby reduce the time of contact between the hydrolytic enzymes and said released PHAs.

In one embodiment, the separating step comprises using at least one of flotative separation, centrifugation or membrane separation.

The disclosed process and/or method may also be carried out in non-aseptic conditions.

### BRIEF DESCRIPTION OF DRAWINGS

The accompanying drawings illustrate a disclosed embodiment and serves to explain the principles of the disclosed embodiment. It is to be understood, however, that the drawings are designed for purposes of illustration only, and not as a definition of the limits of the invention.

FIG. 1 shows a schematic process flow diagram of the PHA production process.

FIG. 2a is a graph showing the change in pH with respect to the change in time (measured in days) in the anaerobic reactor 6 of FIG. 1.

FIG. 2b is a graph showing the change in Oxidation-Reduction Potential (ORP, measured in millivolts, mMV) with respect to the change in time (measured in days) in the anaerobic reactor 6 of FIG. 1.

FIG. 3 is another graph showing the change in the biomass concentration (measured in grams per litre) with respect to the change in time (measure in hours) of the aerobic reactor 8 of FIG. 1.

### DETAILED DESCRIPTION OF DRAWINGS

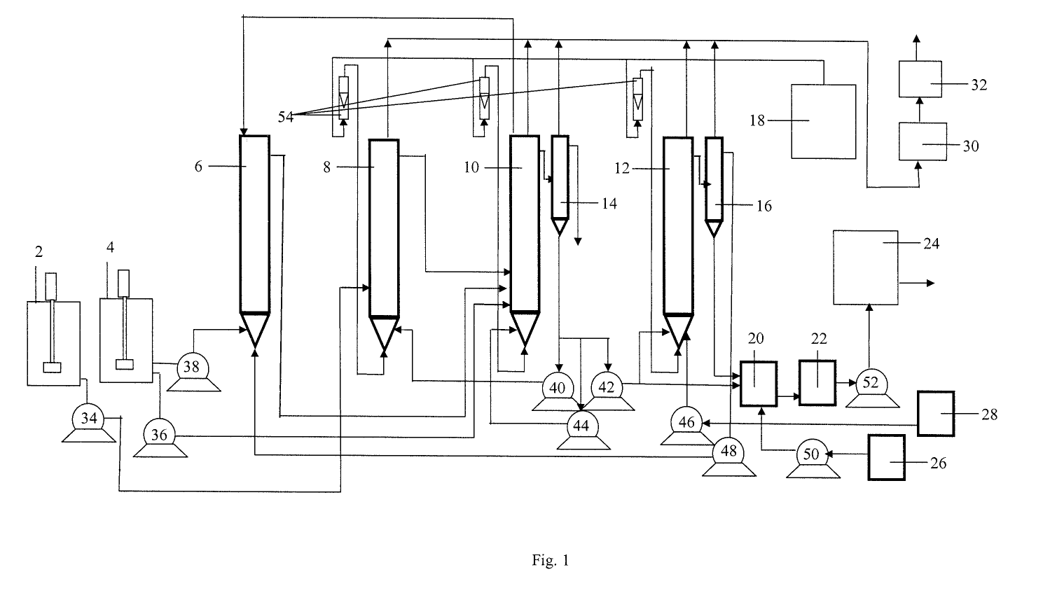

Referring now to FIG. 1, there is shown a system 100 for use in the production of PHA. The system 100 consists of stirring tanks (2, 4), anaerobic reactor 6, aerobic reactors (8, 10, 12), settling tanks (14, 16), air compressor 18, storage tanks (20, 22, 24, 26, 28), a separator 30, granulated activated carbon (GAC) filter 32 pumps (34, 36, 38, 40, 42, 44, 46, 48, 50, 52) and a air flow meter 54.

Two stirring tanks (2, 4) are provided for the storage of inorganic nutrients and organic nutrients respectively. Stirring tank 2 houses the inorganic medium (hereafter “M2”) which is to be supplied to aerobic reactor 8, which preferentially stimulates the growth of microbes adapted for PHA production.

The inorganic medium M2 in stirring tank 2 is prepared as follows: 300 g of glucose is dissolved in 15 L of tap water. 5 L of other solutions, selected from the group consisting of: reject water, solution of inorganic nutrients, solution of organic acids after fermentation, acetic acid, valeric acid, chloric acid and sodium hydroxide, are added to this volume so that the final concentration of carbohydrates in the medium is 20 g/L. 5 L of reject water procured from a municipal wastewater treatment plant containing approximately 1 g volatile fatty acids per litre is further added to the medium. The final concentration of volatile fatty acids is about 5 g/L. If reject water of municipal wastewater plants is not used, inorganic nutrients can be manually added to the medium, to achieve the respective final concentrations:

| Compound | Concentration (gL⁻¹) |
|---|---|
| (NH₄)₂Cl | 2 g |
| KH₂PO₄ | 0.5 |
| CaCl₂•2H₂O | 0.1 |
| MgCl₂•6H₂O | 0.1 |
| Ferric EDTA sodium salt | 0.1 |
| hydrate | |
| MnCl₂•4H₂O | 0.01 |
| Na₂MoO₄•2H₂O | 0.001 |

The pH of the inorganic medium M2 is kept in a range of 6.5 to 8.2. This inorganic medium M2 is then pumped into reactor 8 at a rate of 1 L/hour (24 L/day).

The organic medium M1 in stirring tank 4 is prepared as follows: 300 g of glucose is dissolved in 10 L of tap water. 10 L of biomass suspension (with PHA extracted) is further added to this volume so that final concentration of carbohydrates in this medium is about 20 g/L and the final concentration of protein is about 0.8 g/L (i.e. the final concentration of nitrogen is about 0.12 g/L). This organic medium M1 is then pumped into the anaerobic reactor 6 at a rate 1 L/hour (24 L/day). This organic nutrient medium is also pumped into reactor 10 at a rate of 1 L/hour (24 L/day).

Anaerobic reactor 6 serves as the fermentation reactor, primarily for the production of organic acids, volatile organic compounds and hydrogen gas. Anaerobic reactor 6 has an operating volume of 13 L. To start up the anaerobic reactor 6, it is filled with 6 L of organic medium M1 from stirring tank 4 and 1 L of an anaerobic soil suspension. This soil suspension is made by mixing of 0.5 kg of wet anaerobic soil obtained from wetland or lake shore, with 1 L of tap water. This soil mixture is then allowed to settle for 1 hour and filtered through a 0.1 mm screen in order remove the soil debris. The filtered soil suspension is then added into anaerobic reactor 6.

Anaerobic conditions are maintained in the reactor 6, with the oxidation-reduction potential (ORP) kept lower than −50 mV. Batch cultivation is thereafter continued for 4 days, during which the pH is maintained at around 6-8. During this time, organic compounds are fermented by one or more bacteria species provided in reactor 6 to produce hydrogen, volatile organic compounds and organic acids.

During continuous operation, stirring tank 4 supplies organic medium (hereafter “M1”) to the anaerobic reactor 6 at 1 L/hr. Lysed and/or intact biomass exiting from reactor 12 is recycled into anaerobic reactor 6 at a rate of 1 L/hour (24 L/day). Volatile organic compounds exiting from reactor 10 are recycled into anaerobic reactor 6 at a rate of gas 10 L/hour (240 L/day). The productions of anaerobic fermentation, primarily organic acids, volatile organic compounds and hydrogen, are routed to reactor 10.

Reactor 8 has an operating volume of 6 L. Reactor 8 provides a nutrient deficient phase, whereby microbes which are capable of producing and/or storing PHAs are preferentially selected over microbes which cannot produce or store enough PHA to survive this nutrient deficient phase.

To start up reactor 8, 3 L of organic medium M1, 2.5 L of inorganic medium M2 and 0.5 L of aerobic soil suspension is added to reactor 8. The soil suspension is obtained via the same method as that described above, with the difference being that the soil is aerobic. Batch cultivation is then carried out for 2 days.

When in continuous operation, reactor 8 is supplied by stirring tank 2 with inorganic medium M2 at a rate of 1 L/hr. Reactor 8 also receives biomass pumped from reactor 10 at a rate of 3 L/hr. Air is supplied to reactor 8 at 2 L/min, causing an aeration rate of 0.33 L/L min. Air is supplied from a compressor 18, which is in fluid communication with reactors 8, 10 and 12. The rate of air flow is monitored by a flow meter 54. The microbe suspension in reactor 8, which has been enriched in PHA-producing microbes, is pumped back into reactor 10 at a rate of 4 L/hour.

Reactor 10 has an operating volume of 13 L. Reactor 10 is primary reactor for the production of PHA, through the cultivation of microbes capable of producing and storing PHA. To start up reactor 10, it is filled with 10 L of organic medium M1 from stirring tank 4, 2 L of inorganic medium M2 from stirring tank 2 and it of aerobic soil suspension. The soil suspension is obtained the same way as that for reactor 8. Batch cultivation is subsequently carried out for 2 days.

When in continuous operation, reactor 10 is supplied with organic medium M1 from stirring tank 2 at a rate of 1 L/hr. The rate at which the organic medium is supplied to reactor 10 may be adjusted to obtain a total organic carbon (TOC) concentration at about 500 mg/L. The organic acids, volatile organic compounds and hydrogen, reaction products from reactor 6, are pumped into reactor 10 at a rate of 2 L/hr. It is observed that the growth of the PHA containing microbes is significantly improved by passing the hydrogen gas into reactor 10. It is observed that the growth of the PHA containing microbes is significantly improved by passing the volatile organic compounds into reactor 10. Biomass from reactor 8 is pumped into reactor 10 at a rate of 3 L/hr. Aeration rate in reactor 10 is about 10 times lesser than the aeration rate in reactor 8, i.e., air is supplied at about 0.4 L/min and the resultant aeration rate is 0.03 L/Lmin. Air flow rate may be adjusted as need to achieve a dissolved oxygen concentration of about 0.5 mg/L. A portion of the gas exiting reactor 10, which is enriched in volatile organic compounds, is recycled back to reactor 6 at a rate of gas 10 L/hr (240 L/day).

The suspension from the top of reactor 10 is discharged into a settling tank 14 at a rate of 6 L/hr. Liquid from the top of settling tank 14 is discharged into a drain by gravity. The settled biomass at the bottom of settling tank 14 is pumped into reactor 8 at 3 L/hr, reactor 10 at 3 L/hr and reactor 12 at 0.5 L/hr.

Reactor 12 provides for the extraction of PHA from the microbes. The operating volume of reactor 12 is 13 L. To start up the reactor 12, 11 L of settled biomass from settling tank 14 and 2 L of aerobic, acidic forest soil suspension are added into reactor 12. The soil suspension is obtained by mixing 1 kg of the aerobic, acidic forest soil with 2 L of tap water. The mixture is allowed to settle for an hour and is filtered through a 1 mm screen to remove the soil debris. The resultant soil suspension is then added into reactor 12. The pH of the reactor contents is adjusted to about 4.5 using 0.1M solution of hydrochloric acid. Batch cultivation takes place for 4 days. Thereafter, reactor 12 is rife with micro-organisms capable of releasing extra-cellular enzymes for the hydrolysis of bacterial cell walls.

During the continuous phase, reactor 12 is supplied with biomass pumped from settling tank 14 at a rate of 2 L/hr. 0.1M HCl from storage tank 28 is added continuously into reactor 12 at a rate of 0.01-0.1 L/hr, in order to maintain pH in the range of 4.3-4.6. At this stage, a portion of the microbes undergo lysis and the PHA is extracted from the cell in the form of PHA granules.

The foam at the top of reactor 12 is discharged into a separation tank 16, where the separation of the PHA granules and the cell debris takes place. The extraction of PHA from the cells of the PHA-producing microbes is performed simultaneously with the separation of the PHA granules from the cell debris to minimize interactions between the hydrolytic enzymes and the PHA granules. The separation is performed by flotation of the PHA granules at a pH of about 3.5. A portion of the liquid exiting separation tank 16 is recycled back to reactor 6 at a rate of 1 L/hr.

The crude PHA is then passed to tank 20 at a rate of 0.5 L/hr, where it is subjected to further purification treatment by bleach solution pumped from tank 26. The purified PHA is passed to and stored in tank 22 before it is pumped into the drying and granulation unit 24. The drying may be carried out at 60° C. and the granulation carried at 180° C.

The effluent air from the aerobic reactors, 8, 10 and 12 exits from the top of these reactors at a rate of 0.44 L/min and is thereafter routed to a separator 30 and a granulated activated carbon (GAC) filter 32 for treatment prior to being discharged to the surrounding environment.

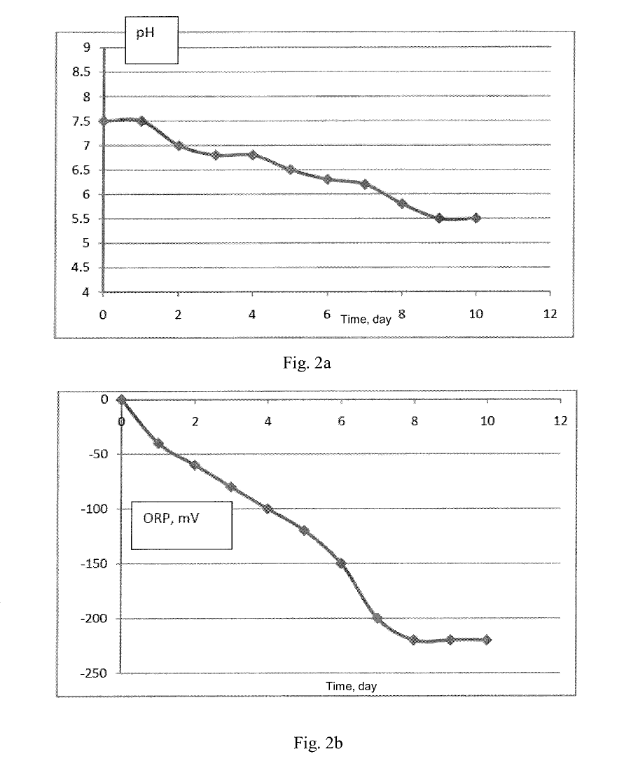

Referring now to FIG. 2a and FIG. 2b, there are respectively depicted two graphs showing the changes in pH and ORP with respect to time occurring in reactor 6. As can be seen from FIG. 2a, pH gradually decreased from 7.5 to about 5.5 over 9 days and the pH did not record a further change on the 10^{th} day. It is postulated that as more organic acids are produced due to the fermentation process, the pH drops accordingly until the net organic acid concentration in reactor becomes substantially constant. This can be due to steady state being reached, where the amount of organic acids being routed to reactor 10 is replaced by newly synthesized organic acids in the fermentation reactor, with no net increase or decrease in organic acid concentration.

In the second graph, it can be seen that the redox potential decreases sharply over the first 8 days and remains fairly constant over the next two days at approximately −220 millivolts. The high negativity of the redox potential is indicative of the anaerobic conditions essential for the fermentation and the production of organic acids and hydrogen. It can be seen that the conditions in the reactor grew more anaerobic with time until a steady state value was reached.

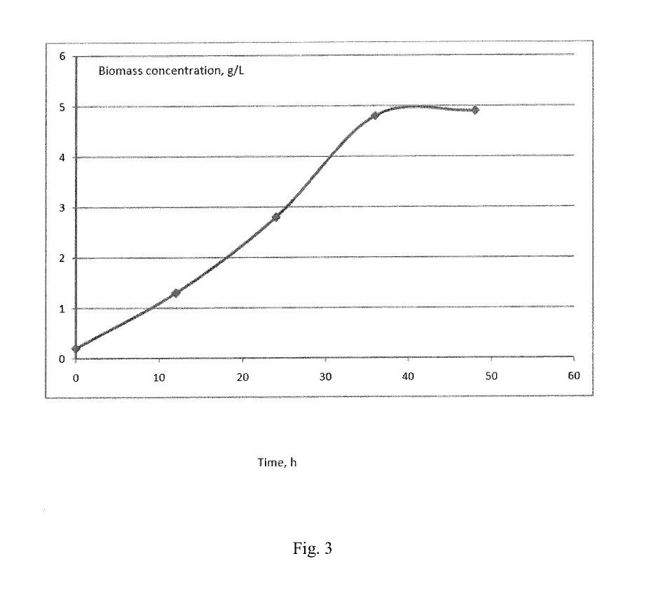

Now referring to FIG. 3, there is depicted a graph showing the change of biomass concentration with respect to time in the reactor 10. As expected, the biomass concentration grows at a fairly constant pace, peaking at about 5 g/L on the 40^{th} hour. The biomass concentration then maintains at a value of about 5 g/L. This concentration is indicative of the maximum microbe population sustainable, wherein further growth is prevented by a limiting factor, such as the rate at which organic nutrients are assimilated.

### Applications

The disclosed methods and processes may be applied in numerous industrial applications, not least in the production of biodegradable plastics as packaging material and as biomaterials for applications in both conventional medical devices and tissue engineering.

The disclosed methods and processes are able to synthesize PHAs into PHAs-containing biomass efficiently and economically. Furthermore, as the disclosed methods and process can employ wastewater or solid organic wastes as the medium for the production of PHAs, these methods reduce cost of raw materials used.

In one embodiment, through the unique combination of anaerobic fermentation, aerobic biomass growth, microaerophilic biosynthesis of PHAs, microbial hydrolysis of biomass containing PHAs, and flotative concentration of PHAs, the disclosed method is further capable of and ensuring a continuous production of PHAs.

Furthermore, the disclosed methods avoid the need to use flammable and toxic organic solvents in the chemical extraction of PHAs. Advantageously, the disclosed method is capable of minimizing environmental pollution.

The disclosed methods and processes are also not restricted to the use of pure cultures of bacteria. Advantageously, the methods and processes can be carried out in aseptic conditions. More advantageously, large amount of resources does not have to be expended to keep the process conditions sterile of free from foreign micro-organisms.

The disclosed method of increasing the proportion of PHAs-producing microbes relative to non-PHAs producing microbes in a culture media containing both PHAs-producing microbes and non-PHAs producing microbes is not limited to being used in batch processes. Advantageously, the disclosed methods do not result in the micro-organisms frequently altering their natural cellular behavior due to cellular starvation, which when occurs, may undesirably result in the growth rate of the micro-organisms, including PHAs producing micro-organisms being adversely affected. Hence, the method of the present disclosure may result in higher rate of PHA production due to the absence of disturbances in cell physiology during the famine-feast cycles that are carried out in known processes.

More advantageously, the disclosed methods can be used in continuous processes which increase the overall processing efficiency.

It will be apparent that various other modifications and adaptations of the invention will be apparent to the person skilled in the art after reading the foregoing disclosure without departing from the spirit and scope of the invention and it is intended that all such modifications and adaptations come within the scope of the appended claims.

1. A process for producing polyhydroxyalkanoate (PHA) comprising the steps of:
culturing a biomass containing PHA-producing microbes in a culture media;and
hydrolyzing said PHAs-producing microbes using microorganisms selected to release PHAs from the PHAs-containing microbes.

2. A process as claimed in claim 1, wherein said culture media comprises hydrogen gas.

3. A process as claimed in claim 2, comprising the step of injecting a stream of hydrogen gas into said culture medium.

4. A process as claimed in any one of the preceding claims, comprising the step of providing volatile organic compounds to said culture medium.

5. A process as claimed in claim 4, comprising the step of injecting a gas comprising volatile organic compounds into said culture medium.

6. The process as claimed in claim 4 or 5, comprising the step of maintaining the supply of volatile organic compounds in the culture media at a mass ratio in the range of from 0.5 to 5 to the quantity of PHAs produced.

7. The process as claimed in any of the preceding claims, wherein the culture media comprises a carbon nutrient source.

8. The process as claimed in claim 7, wherein the carbon nutrient source comprises carbohydrates.

9. The process as claimed in claim 2 or 3, comprising the step of maintaining the supply of hydrogen in the culture media at mass ratio in the range of from 0.01 to 0.1 of hydrogen to the quantity of PHAs produced.

10. The process as claimed in any one of the preceding claims, comprising the step of maintaining the supply of carbohydrates in the culture media at mass ratio in the range of from 1 to 5 of carbohydrates to the mass of PHAs produced.

11. The process as claimed in any one of the preceding claims, comprising the step of maintaining the supply of at least one of organic acids and lipids in the culture media at mass ratio in the range of from 0.5 to 5 of organic acids or lipids to the mass of PHAs produced.

12. The process as claimed in any one of the preceding claims, comprising the step of maintaining the pH of the culture media in the range of from 6 to 8.

13. The process as claimed in claim 12, wherein the step of maintaining the pH of the culture media comprises providing an organic acid to said culture media.

14. The process as claimed in any of the preceding claims comprising the step of maintaining generally aerobic conditions in said culture media during said culturing step.

15. The process as claimed in claim 14, wherein the culture media comprises from 0.1 mgL⁻¹ to 1 mgL⁻¹ of dissolved oxygen during said culturing step.

16. The process as claimed in any one of the preceding claims, comprising the step of maintaining the culture media at from 100 mgL⁻¹ to 1000 mgL⁻¹ of organic carbon during said culturing step.

17. The process as claimed in any one of the preceding claims, wherein said culturing step comprises culturing said PHA-producing microbes in a culturing zone in which said hydrogen gas is substantially homogenously dispersed throughout said culture media.

18. The process as claimed in any one of the preceding claims, wherein before said culturing step, the process comprises the step of fermenting a population of PHA-producing microbes in a microbial fermentation zone.

19. The process as claimed in claim 18, wherein the microbial fermentation zone is charged with a natural source of microbes that contain PHA-producing microbes.

20. The process as claimed in claim 18 or claim 19, wherein the microbial fermentation zone is maintained at substantially anaerobic conditions.

21. The process as claimed in any one of claims 18 to 20, comprising the step of producing hydrogen gas in said microbial fermentation zone.

22. The process as claimed in any one of claims 18 to 21, comprising the step of obtaining the volatile organic compounds from said microbial fermentation zone.

23. The process as claimed in claim 22, wherein the obtaining step comprises the step of removing a vapor phase above said fermentation zone.

24. The process as claimed in any one of claims 18 to 21, comprising the step of maintaining the microbial fermentation zone at a pH in the range of 5 to 8.

25. The process as claimed in any one of claims 18 to 23, comprising the step of maintaining the microbial fermentation zone at a reduction potential of from −50 mV to −400 mV.

26. The process as claimed in any one of the preceding claims, wherein after said culturing step, the process comprises the step of extracting PHA from said cultured PHA-producing microbes.

27. The process as claimed in claim 25, wherein said extraction step comprises the step of hydrolyzing the PHA-producing microbes to release said PHAs.

28. The process as claimed in claim 26, wherein the step of hydrolyzing comprises the use of using microorganisms that release enzymes that hydrolyzes bacterial cell walls.

29. The process as claimed in claim 27, wherein the microorganisms that release enzymes that hydrolyzes bacterial cell walls are fungi selected from the group consisting of fungi from the genera Absidia, Agaricus, Aspergillus, Chaetomium, Fusarium, Neurospora, Penicillium, Phanerophaete, Phialophora, Pleurotus, Rhizoctonia and Trichoderma.

30. The process as claimed in any one of claims 26 to 28, wherein before said hydrolyzing step, said PHA-producing microbes are substantially separated from the biomass.

31. The process as claimed in any one of claims 26 to 29, wherein said extraction step is undertaken in an extraction zone at generally acidic conditions.

32. The process as claimed in claim 30, wherein said acidic conditions of said extraction zone are at a pH within the range of from 2 to 5.

33. The process as claimed in any one of claims 28 to 31, further comprising the step of separating said released PHAs from said microbes.

34. The process as claimed in claim 32, wherein the extracting step is performed simultaneously with the separating step to thereby reduce the time of contact between the hydrolytic enzymes and said released PHAs.

35. The process as claimed in claim 29, comprising the step of introducing an oxidant to said extraction zone.

36. A method of extracting PHAs from PHAs-containing microbes, the method comprising the steps of:
hydrolyzing the cell walls of the PHAs-containing microbes to release PHAs from the PHAs-containing microbes; and
separating said released PHAs from said microbes.

37. The method of claim 36, wherein the hydrolyzing step comprises the step of using microorganisms that release enzymes that hydrolyzes bacterial cell walls.

38. The method as claimed in claim 37, wherein the microorganisms that release enzymes that hydrolyzes bacterial cell walls are fungi selected from the group consisting of fungi from the genera Absidia, Agaricus, Aspergillus, Chaetomium, Fusarium, Neurospora, Penicillium, Phanerophaete, Phialophora, Pleurotus, Rhizoctonia and Trichoderma.

39. The method as claimed in any one of claims 36 to claim 38, wherein the hydrolysing step is performed simultaneously with the separating step to thereby reduce the time of contact between the hydrolytic enzymes and said released PHAs.

40. The method of any one of claims 36 to 38, wherein the separating step comprises using at least one of flotative separation, centrifugation or membrane separation.

41. A process for producing polyhydroxyalkanoate (PHA) comprising the steps of:
(a) incubating a culture media containing PHAs-producing microbes and non-PHAs producing microbes in a selection zone under conditions to enable faster propagation of the PHAs-producing microbes relative to the non-PHAs producing microbes, wherein said incubating is undertaken for a period of time to produce culture media having more PHA microbes relative to the non-PHA producing microbes;
(b) providing said culture media produced in said incubating step (a) to a culturing zone to culture the PHA-producing microbes in a culture media containing hydrogen gas;
(c) providing said PHA produced in said providing step (a) to an extraction zone in which said PHA producing microbes are hydrolyzed by microorganisms to release PHAs from the PHAs-containing microbes; and
(d) isolating said PHAs from said culture media.

42. The process as claimed in claim 41, wherein at least one of steps (a) to (d) is carried out under non-aseptic conditions.

43. The process as claimed in claim 41 or claim 42, comprising the step of returning at least a portion of the PHA-producing microbes produced in step (b) to the selection zone.
